(19) United States
(12) Patent Application Publication
(10) Pub. No.: US 2021/0208170 A1
(43) Pub. Date: Jul. 8, 2021

(54) A METHOD FOR REGULATED MANIPULATION OF A BIOLOGICAL SAMPLE AND A SYSTEM THEREOF

(71) Applicant: SPOVUM TECHNOLOGIES PRIVATE LIMITED [IN/IN], Bangalore, Karnataka (IN)

(72) Inventors: Santosh DB BHARGAV, Bangalore (IN); Ramnath TJ BHATTA, Madurai (IN)

(21) Appl. No.: 17/058,609

(22) PCT Filed: May 19, 2019
(86) PCT No.: PCT/IN2019/050407

§ 371 Date: Nov. 24, 2020

(30) Foreign Application Priority Data
May 25, 2018 (IN) 201841019672

(51) Int. Cl. G01N 35/00; G01N 33/543; G02B 21/32; G01N 35/04

(52) U.S. Cl. CPC G01N 35/0099; G01N 33/54366; G01N 2035/042; G01N 35/04; G02B 21/32

(57) ABSTRACT

The invention provides a method for regulated manipulation of a biological sample. The method includes selecting the biological sample, initiating a first manipulation on the selected biological sample and performing a second manipulation, subsequent to the first manipulation. The invention also provides an automated system for regulated manipulation of a biological sample. The system includes a stage for positioning the sample, a sensing means coupled to the stage, a first manipulation device positioned in the plane of the stage for a first manipulation of the biological sample, a second manipulation device positioned in-plane or out of plane of the stage for a second manipulation of the biological sample and an analyzer coupled to each of the first manipulation device and the second manipulation device. The coupling enables the analyzer to independently regulate the first manipulation device, the second manipulation device and a combination thereof.

### FIELD OF INVENTION

The invention generally relates to the field of biomedical engineering and particularly to a method and a system for regulated manipulation of a biological sample.

### BACKGROUND

Holding of a sample and subsequent manipulation, by isolating the sample from a given environment has numerous medical and scientific applications. Examples of manipulation include, but are not limited to, squeezing, gripping, grasping, rolling, stretching, bending, piercing, injecting, probing and tearing. Examples of samples include, but are not limited to, biological cells, tissues, scaffolds, tissue-scaffolds, biopsies, zygotes, organelles, platelets, sand-particles, amorphous, crystals and crystalline structures. The said manipulation can be achieved by a manipulator, constructed specifically to perform the said manipulation. Each of the manipulation stated herein above requires precise handling of the manipulator. Manipulators can be rigid, elastic or partially resilient.

Various manipulators have been designed for gripping samples. U.S. Pat. No. 9,340,762, assigned to Yu Sun, Zhe Lu, Clement Leung, Xuping Zhang, discloses a system and a method for automated, computer-controlled, tracking and manipulation of a specimen including sperm and oocytes. The system and method of the invention are suited for tracking and manipulating sperm, particularly for Intracytoplasmic Sperm Injection (ICSI) procedures. The invention utilizes a micropipette for manipulation of the specimen. One of the disadvantages of using micropipette is a significant amount of loss of specimen during handling. Other disadvantages include handling difficulty and complex arrangement of devices. Hence, there is a need for a regulated manipulation system that is simple, robust and offers ease of use.

### BRIEF DESCRIPTION OF DRAWINGS

So that the manner in which the recited features of the invention can be understood in detail, some of the embodiments are illustrated in the appended drawings. It is to be noted, however, that the appended drawings illustrate only typical embodiments of this invention and are therefore not to be considered limiting of its scope, for the invention may admit to other equally effective embodiments.

FIG. 1 shows a system for regulated manipulation of a biological sample, according to an embodiment of the invention.

FIG. 2a shows a first manipulation device, according to an embodiment of the invention.

FIG. 2b shows an exploded view of the manipulation device, according to an embodiment of the invention.

FIG. 3a shows the first manipulation device before the manipulation of a biological sample, according to an embodiment of the invention.

FIG. 3b shows the first manipulation device after the manipulation of a biological sample, according to an embodiment of the invention.

### SUMMARY OF THE INVENTION

One aspect of the invention provides a method for regulated manipulation of a biological sample. The method includes selecting the biological sample, initiating a first manipulation on the selected biological sample and performing a second manipulation, subsequent to the first manipulation. The first manipulation and the second manipulation are regulated.

Another aspect of the invention provides an automated system for regulated manipulation of a biological sample. The system includes a stage for positioning the sample, a sensing means coupled to the stage, a first manipulation device positioned proximal to the sample for a first manipulation of the biological sample, a second manipulation device opposingly positioned the first manipulator for a second manipulation of the biological sample and an analyzer coupled to each of the first manipulation device and the second manipulation device. The coupling enables the analyzer to independently regulate the first manipulation device, the second manipulation device and a combination thereof.

### DETAILED DESCRIPTION OF THE INVENTION

Various embodiments of the invention provide a method for regulated manipulation of a biological sample. The method includes selecting the biological sample, initiating a first manipulation on the selected biological sample and performing a second manipulation, subsequent to the first manipulation. The method described herein briefly shall be explained in detail.

Initially, a biological sample is selected. Examples of the biological sample include, but are not limited to, an oocyte, a zygote, an embryo, sperm or any other non-motile specimen. Upon selection of the biological sample, a first manipulation is initiated. Examples of the first manipulation include but are not limited to immobilization, squeezing, gripping, grasping, rolling, stretching, bending, piercing, injecting, probing, tearing or a combination thereof. In one example of the invention, the first manipulation is an immobilization. The step of immobilization includes identifying the sample, positioning a device around the identified sample and actuating the device to releasably immobilize the sample. Subsequent to the first manipulation, a second manipulation is performed. Examples of the second manipulation include but are not limited to squeezing, gripping, grasping, rolling, stretching, bending, piercing, injecting, probing, tearing or a combination thereof. The first manipulation and the second manipulation described herein are regulated. The regulation is achieved by automation of the first manipulation and the second manipulation.

The method, as described hereinabove, is achieved by using an automated system for regulated manipulation of a biological sample. The system shall be described and illustrated through the accompanying drawings.

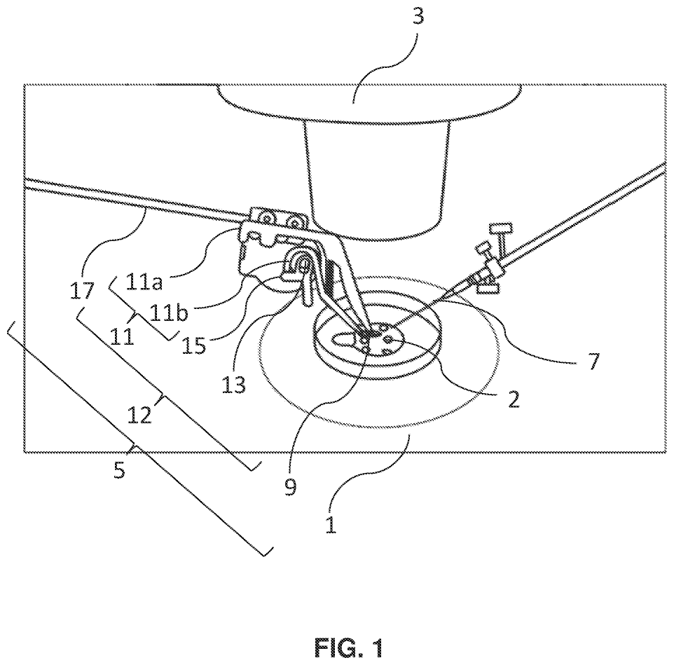

FIG. 1 shows a system for regulated manipulation of a biological sample, according to an embodiment of the invention. The system includes a stage 1 for positioning the biological sample 2. A sensing means 3 is coupled to the stage 1. A first manipulation device 5 is positioned proximal to the biological sample 2 for a first manipulation. A second manipulation device 7 is opposingly positioned to the first manipulation device 5 and is configured for a second manipulation of the biological sample 2. An analyzer (not shown) is coupled to each of the first manipulation device 5 and the second manipulation device 7. The system described hereinabove briefly shall be explained in detail.

The system includes a stage 1 for positioning the biological sample 2. Examples of the biological sample include but are not limited to an oocyte, a zygote, an embryo, sperm or any other non-motile specimen. The stage 1 is capable of translational motion along the x, y and the z axes. The stage 1 is also capable of a rotational motion in the x-y plane. The system further includes a sensing means 3 coupled to the stage 1. The sensing means 3 is capable of sensing the location and other physical properties of the sample 2. The sensing means 3 can be an objective means with a lens or a system of lenses coupled to an imaging device. Examples of the imaging device include, but are not limited to, an arrangement of lenses, a camera or a charge coupled device camera.

A first manipulation device 5 is positioned proximal to the biological sample 2 for performing a first manipulation. In one example of the invention, the first manipulation is an immobilization of the biological sample. The step of immobilization includes holding the sample 2 at a given location within a sample holder containing the sample 2. The first manipulation device includes a micro gripper 9 and a first actuator 12 coupled to the micro gripper 9. The micro gripper 9 is made up of a material that makes it robust and less fragile.

In one example of the invention, the gripper is made up of elastomers. The first actuator 12 described herein is a compliant mechanism-based actuator. The first actuator 12 includes a gripper holder 11, a motor 13 and an actuating pin 15. The gripper holder 11 is made up of a material that is stiffer than that of gripper 9 since the gripper holder 11 actuates the gripper 9. The gripper 9 and the gripper holder 11 are manufactured by a technique that includes but is not limited to microfabrication, electro-discharge machining (EDM), micromachining, lithography, nanofabrication, surface micromachining, 3D-prinitng, molding, casting, stamping, sheet metal forming, LASER-cutting, punching, milling, stencil-cutting, etching, adhesive bonding or a combination thereof.

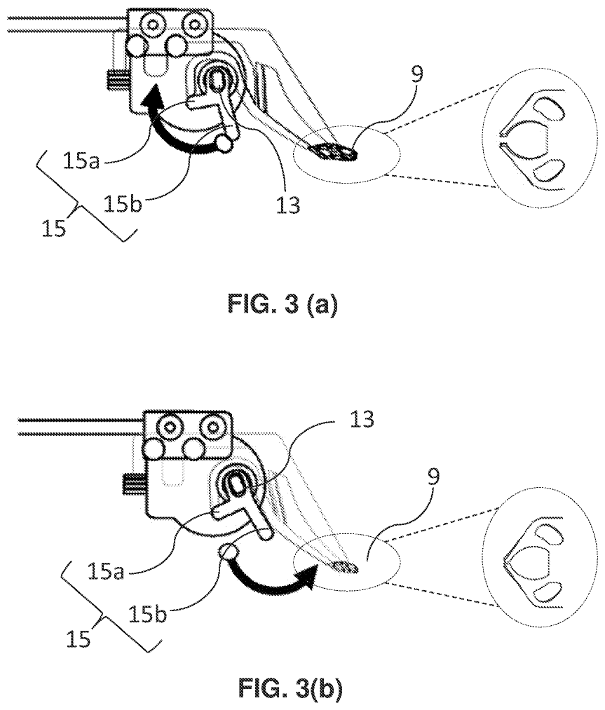

The gripper holder 11 has a first end 11a and a second end 11b. The first manipulation device is additionally provided with a connecting rod 17 to support the first actuator 12. The connecting rod 17 is attached to the first end 11a of the gripper holder 11 using lock pins 19. The actuating pin 15 is coupled to the second end 11b of the gripper holder 11. Upon actuation of the motor 13, the actuating pin 15 comes in contact with the second end 11b of the gripper holder 11 to enable movement of the micro gripper jaws, which in turn enables immobilization of the biological sample. The motor 13 is configured for a bi directional rotation, namely, a clock wise rotation and an anti-clockwise rotation. In one example of the invention, the motor 13 is a stepper motor and each step corresponds to about 0.175 degree rotation. The movement of the micro gripper jaws is explained in conjunction with FIG. 3.

A second manipulation device 7 is positioned in-plane or out of plane of the stage for a second manipulation of the biological sample 2. The second manipulation includes but is not limited to squeezing, gripping, grasping, rolling, stretching, bending, piercing, injecting, probing, tearing or a combination thereof. The second manipulation device 7 includes an intra cytoplasmic injection device and a second actuator coupled to the intra cytoplasmic injection device 7. The operation of the second actuator can be manual, motor driven, hydraulic, pneumatic and/or a combination thereof.

An analyzer (not shown) is coupled to each of the first manipulation device 5 and the second manipulation device 7. The coupling enables the analyzer to independently regulate the first manipulation device 5, the second manipulation device 7 and a combination thereof. The analyzer is coupled independently to each of the stage 1, the objective means 3, the first manipulation device 5 and the second manipulation device 7. The analyzer includes a user input, a computational engine and a display means. In one embodiment of the invention, the system accepts a user input to actuate the compliant mechanism-based gripper to manipulate the biological sample. The input can be through a joystick, touch-screen display, human voice input, gestures or a combination thereof. The computational engine is configured for receiving a first set of instructions from the stage 1 and the objective means 3, transmitting a second set of instructions to the first actuator 12 and transmitting a third set of instruction to the second actuator. The first set of instructions corresponds to properties including position, orientation, size of the biological sample or a combination of them. The second set of instructions corresponds to a first manipulation of the biological sample. The third set of instructions corresponds to a second manipulation of the biological sample. Further, the system is also configured for retrievably storing a plurality of information obtained during manipulation. The information described herein, includes, but is not limited to, automatically captured data like timestamps, the actions performed, the image and/or videos captured and the data manually entered by the user during the process including patient ID, health details of the biological sample and the like data. The display means is capable of displaying the first manipulation and the second manipulation.

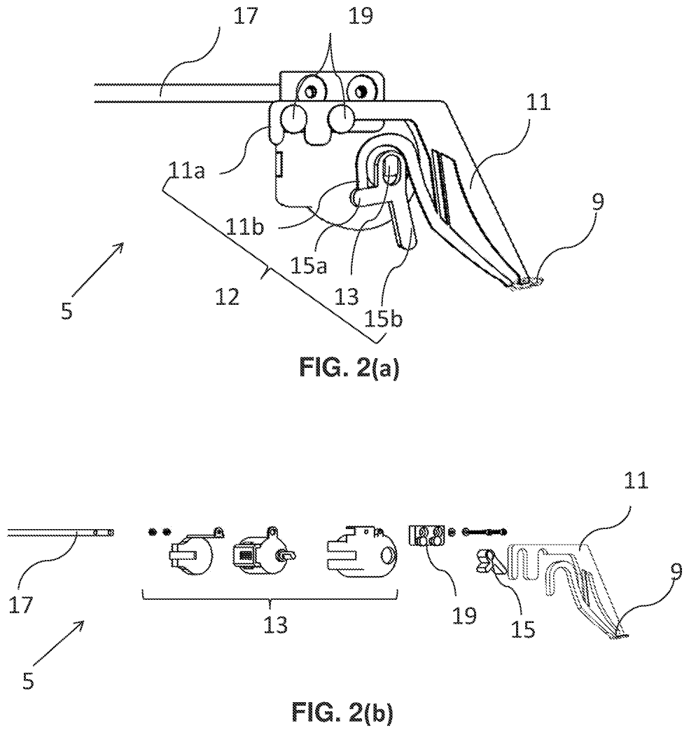

FIG. 2a shows a manipulation device, according to an embodiment of the invention. The first manipulation device includes a micro gripper 9 and a first actuator 12 coupled to the micro gripper 9. The first actuator 12 described herein is a compliant mechanism-based actuator. The first actuator 12 includes a gripper holder 11, a motor 13 and an actuating pin 15. The gripper holder 11 is having a first end 11a and a second end 11b. The first manipulation device is additionally provided with a connecting rod 17 to support the first actuator 12. The connecting rod 17 is attached to the first end 11a of the gripper holder 11 using lock pins 19. The actuating pin 15 is coupled to the second end 11b of the gripper holder 11. Upon actuation of the motor 13, the actuating pin 15 comes in contact with the second end 11b of the gripper holder 11 to enable movement of the micro gripper jaws, which in turn enables immobilization of the biological sample. The motor 13 is configured for rotation in clock wise and anti-clockwise directions. In one example of the invention, the motor 13 is a stepper motor and each step corresponds to about 0.175 degree rotation. FIG. 2b shows an exploded view of the manipulation device, according to an embodiment of the invention.

FIG. 3 generally shows the various stages of positioning of the first manipulation device, according to an embodiment of the invention.

FIG. 3a shows the first manipulation device before the manipulation of a biological sample, according to an embodiment of the invention. Upon clockwise rotation of the motor 13, the first end 15a of the actuating pin 15 moves backward as shown in the figure. The backward motion of the actuating pin 15a results in opening of the jaws of the gripper 9. FIG. 3b shows the first manipulation device after manipulation of a biological sample, according to an embodiment of the invention. Upon anti-clockwise rotation of the motor 13, the second end 15b of the actuating pin 15 moves forward as shown in the figure. The forward motion of the actuating pin 15b results in closing of the jaws of the gripper 9. The opening/closing of the jaws enables manipulation of the biological sample. The manipulation described herein includes but is not limited to immobilization, squeezing, gripping, grasping, rolling, stretching, bending, piercing, injecting, probing, tearing or a combination thereof.

In an alternate embodiment of the invention, the first manipulation and the second manipulation can be performed by the first manipulation device.

### INDUSTRIAL APPLICABILITY

The method and system of regulated manipulation of a biological sample, as described herein, is applied broadly in the area of reproductive biology. The method and the system can be applied to induce artificial insemination. Additionally, the method and the system can also be applied to study the cell at various levels of formation, i.e., from the stage of fertilization to the stage of multiplication of the cells. In one specific implementation, the method was applied to artificial insemination of an egg.

The ovum selected for insemination is taken in a temperature regulated dish and is placed on the stage. The stage is then positioned to bring the egg within the field of view of the sensing means. The first manipulator is then aligned to bring the jaws of the micro gripper in close proximity to the egg. Upon positioning proximal to the egg, the first manipulator is actuated through the actuating means to immobilize the egg. The analyzer coupled to the first manipulator enables the first manipulator to hold the egg in position without exerting additional force on the egg. The analyzer is capable of calculating the amount of force required, to hold the egg in position without altering the physical properties of the egg, based on pre-stored simulated parameters. Subsequent to immobilizing the egg in position, the second manipulator is opposingly positioned to the first manipulator. The second manipulator is an intercytoplasmic injection device holding a pre-determined volume of seminal fluid containing sperms. The second manipulator is then aligned to bring the tip of the injection device in close proximity to the immobilized egg. Upon alignment of the second manipulator, which is confirmed through the sensing means, the second manipulator is actuated to inject the fluid containing the sperms into the egg. The analyzer coupled to the first manipulator, the second manipulator and the sensing means is configured to capture a static image, a dynamic image or a video of the various stages of the first manipulation and the second manipulation. The artificial insemination process, as described herein can be achieved with minimal human intervention, thereby reducing the inherent errors of rupture of egg, loss of temperature, which are the predominant factors for failure of successful insemination in the procedures, existing in the art.

Thus, the invention provides a method and an automated system for regulated manipulation of a biological sample. The system has advantages in terms of ease of use, robustness, automatic data collection and data processing. Further, the system provides multi-point immobilization of the biological sample.

The foregoing description of the invention has been set merely to illustrate the invention and is not intended to be limiting. Since modifications of the disclosed embodiments incorporating the spirit and substance of the invention may occur to person skilled in the art, the invention should be construed to include everything within the scope of the appended claims and equivalents thereof.

We claim:

1. A method for regulated manipulation of a biological sample, the method comprising of:
selecting the biological sample;
initiating a first manipulation on the selected biological sample; and
performing a second manipulation, subsequent to the first manipulation;
wherein the first manipulation and the second manipulation are regulated.

2. The method as claimed in claim 1, wherein the biological sample selected can be an oocyte, a zygote or an embryoor sperm or any other non-motile specimen.

3. The method as claimed in claim 1, wherein the first manipulation is at least one of immobilization, squeezing, gripping, grasping, rolling, stretching, bending, piercing, injecting, probing, tearing or a combination thereof.

4. The method as claimed in claim 1, wherein the step of immobilization includes identifying the sample, positioning a device around the identified sample, and actuating the device to releasably immobilize the sample.

5. The method as claimed in claim 1, wherein the second manipulation is at least one of squeezing, gripping, grasping, rolling, injection, stretching, bending, piercing, injecting, probing, tearing or a combination thereof.

6. The method as claimed in claim 1, wherein the regulation is achieved by automation of the first manipulation, second manipulation and/or a combination thereof.

7. A system for regulated manipulation of a biological sample, the system comprising of:
a stage for positioning the sample;
a sensing means coupled to the stage;
a first manipulation device positioned proximal to the biological sample;
a second manipulation device opposingly positioned the first manipulation device; and
an analyzer coupled to each of the first manipulation device and the second manipulation device, wherein the coupling enables the analyzer to independently regulate the first manipulation device, the second manipulation device and a combination thereof.

8. The system as claimed in claim 7, wherein the stage is capable of translational motion along the x, y and the z axes.

9. The system as claimed in claim 7, wherein the stage is further capable of a rotational motion in the x-y plane.

10. The system as claimed in claim 7 wherein the sensing means is capable of sensing the location and other physical properties of the sample.

11. The system as claimed in claim 7, wherein the sensing means can be an objective means with a lens or a system of lenses coupled to an imaging device, wherein the imaging device can be a camera, a charge coupled device camera.

12. The system as claimed in claim 7, wherein the first manipulation device comprises of a micro gripper and a first actuator coupled to the micro gripper.

13. The system as claimed in claim 7, wherein the first actuator comprises of a gripper holder, a motor and an actuating pin.

14. The system as claimed in claim 7, wherein the first actuator is a compliant mechanism-based actuator.

15. The system as claimed in claim 7, wherein the second manipulation device comprises of an intra cytoplasmic injection device and a second actuator coupled to the intra cytoplasmic injection device.

16. The system as claimed in claim 7, wherein the operation of the second actuator can be manual, motor driven, hydraulic, pneumatic and/or a combination thereof.

17. The system as claimed in claim 7, wherein the analyzer is coupled independently to each of the stage, the objective means, the first actuator and the second actuator.

18. The system as claimed in claim 7, wherein the analyzer comprises of a computational engine, a display means and user input.

19. The system as claimed in claim 7, wherein the computational engine is configured for
receiving a first set of instructions from the stage and the objective means;
transmitting a second set of instruction to the first actuator; and
transmitting a third set of instruction to the second actuator.

20. The system as claimed in claim 7, wherein the first set of instructions comprises of properties selected from position of the biological sample, orientation of the biological sample, size of the biological sample or a combination of them.

21. The system as claimed in claim 7, wherein the second set of instructions comprises of a first manipulation of the biological sample.

22. The system as claimed in claim 7, wherein the third set of instructions comprises of a second manipulation of the biological sample.

23. The system as claimed in claim 7, wherein the first manipulation and the second manipulation is either identical or distinct.

24. The system as claimed in claim 7, wherein the first manipulation and the second manipulation is at least one of squeezing, gripping, grasping, rolling, injection, stretching, bending, piercing, injecting, probing, tearing or a combination thereof.

25. The system as claimed in claim 7, wherein the display means is capable of displaying the first manipulation and the second manipulation.
